(19) United States
(12) Patent Application Publication
(10) Pub. No.: US 2015/0208963 A1
(43) Pub. Date: Jul. 30, 2015

(54) MICRO SENSING SYSTEM FOR DETECTING NEUROTROPHIC FACTORS

(71) Applicant: KOREA INSTITUTE OF SCIENCE AND TECHNOLOGY, Seoul (KR)

(72) Inventors: Hyunjoo Jenny LEE, Seoul (KR); Il-Joo CHO, Seoul (KR); Eui Sung YOON, Seoul (KR); Nakwon CHOI, Seoul (KR)

(73) Assignee: KOREA INSTITUTE OF SCIENCE AND TECHNOLOGY, Seoul (KR)

(21) Appl. No.: 14/534,378

(22) Filed: Nov. 6, 2014

(30) Foreign Application Priority Data
Jan. 24, 2014 (KR) 10-2014-0009180

(51) Int. Cl. A61B 5/145; A61B 5/00

(52) U.S. Cl. CPC A61B 5/14514; A61B 5/685; A61B 5/4064

(57) ABSTRACT

A micro sensing system for real-time sensing a neurotrophic factor included in a body fluid includes a body, a neurotrophic factor channel formed in the body to allow a fluid flow therein, and a biosensor formed at the body to sense the neurotrophic factor, wherein the body fluid is extracted in a living body through the neurotrophic factor channel, and wherein the biosensor is disposed on a path of the neurotrophic factor channel to directly contact a body fluid flowing through the neurotrophic factor channel and senses a concentration of a neurotrophic factor in the body fluid.

### CROSS-REFERENCE TO RELATED APPLICATION

This application claims priority to Korean Patent Application No. 10-2014-0009180, filed on Jan. 14, 2014, and all the benefits accruing therefrom under 35 U.S.C. §119, the contents of which in its entirety are herein incorporated by reference.

### BACKGROUND

1. Field

The present disclosure relates to a micro sensing system, and more particularly, to a micro sensing system for detecting neurotrophic factors, which is included in a body fluid such as a brain fluid.

[Description about National Research and Development Support]This study was supported by the Original Technology R&D Project for Brain Science of Ministry of Science, ICT and Future Planning, Republic of Korea (Project No. 2013076618) under the superintendence of National Research Foundation of Korea.

2. Description of the Related Art

Recently, studies for applying nerve stimulation and a resultant signal to treat diseases and reveal brain activities are in active progress. In particular, in animal testing, studies for revealing neurotrophic factors in relation to behaviors or diseases of an animal by observing the change of concentration of various neurotrophic factors in relation to behaviors or diseases of an animal are spotlighted.

In an existing technique, a micro dialysis system fabricated using a glass tube is used for extracting neurotrophic factors, the neurotrophic factor is collected by an external storage unit, and a concentration of a selected neurotrophic factor is measured using a sensor.

However, in the existing technique, the glass tube having a large size may damage the brain when being inserted into the brain.

In addition, since brain neurotrophic factors extracted from the brain are very little and a fluid flowing through the glass tube is very slow, the brain neurotrophic factors should be extracted for a very long time when they are collected in a storage unit at the outside.

Therefore, even though a concentration of a specific brain neurotrophic factor changes due to any disease, the corresponding factor is extracted in the storage unit at the outside well after the change, and thus it is difficult to establish real-time interrelationship between the disease and the neurotrophic factor in relation, which may deteriorate the reliability of the test.

### SUMMARY

The present disclosure is directed to providing a micro sensing system, which may minimize a brain damage by using a subminiature micromachining technique and sense a change of a neurotrophic factor in real time even though the neurotrophic factor changes by a very little amount.

In one aspect, there is provided a micro sensing system for sensing a neurotrophic factor included in a body fluid, which includes: a body; a neurotrophic factor channel formed in the body to allow a fluid flow therein; and a biosensor formed at the body to sense the neurotrophic factor, wherein the body fluid is extracted in a living body through the neurotrophic factor channel, and wherein the biosensor is disposed on a path of the neurotrophic factor channel to directly contact a body fluid flowing through the neurotrophic factor channel and senses a concentration of a neurotrophic factor in the body fluid.

In an embodiment, the body may include a storage unit formed on a path of the neurotrophic factor channel to store the body fluid flowing along the neurotrophic factor channel, and the biosensor may be integrated in the storage unit.

The neurotrophic factor channel and the storage unit may be formed simultaneously by means of deep etching.

In addition, a cover may be provided to close upper portions of the neurotrophic factor channel and the storage unit, and a plurality of protrusions for preventing the cover from collapsing may be formed at the storage unit.

The micro sensing system may further include a buffer solution channel formed in the body to allow a buffer solution to flow into the living body, and a pressure in the living body may increase by the buffer solution flowing into the living body, and thus the body fluid may flow into the neurotrophic factor channel.

In an embodiment, the body may include: a probe body extending to be inserted into the living body; and a main body formed at a rear end of the probe body to be located out of the living body, wherein the neurotrophic factor channel may extend from the probe body to the main body, and wherein the biosensor may be disposed at the main body.

The biosensor may sense in real time a concentration of the neurotrophic factor in the body fluid which continuously flows in the neurotrophic factor channel.

In addition, the biosensor may be a chemical sensor, an electric sensor or a resonance sensor.

### BRIEF DESCRIPTION OF THE DRAWINGS

FIG. 1 is a perspective view showing a micro sensing system according to an embodiment of the present disclosure.

FIG. 2 is a cross-sectional view, taken along the line A-A′ of FIG. 1.

FIG. 3 is a cross-sectional view, taken along the line B-B′ of FIG. 1.

FIG. 4 is a cross-sectional view, taken along the line C-C′ of FIG. 1.

FIG. 5 is a diagram showing a channel formed at a body.

FIGS. 6A to 6D are diagrams for illustrating a method for forming the structure of FIG. 2.

FIGS. 7A to 7E are diagrams for illustrating a method for forming the structure of FIG. 3.

FIGS. 8A, 8A′, 8A″, 8B to 8D are diagrams for illustrating a method for forming the structure of FIG. 4.

### DETAILED DESCRIPTION

Hereinafter, an embodiment of the present disclosure will be described with reference to the accompanying drawings. Even though the present disclosure is described based on the embodiment depicted in the drawings, this is just an example, and the technical spirit, essences and operations of the present disclosure are not limited thereto.

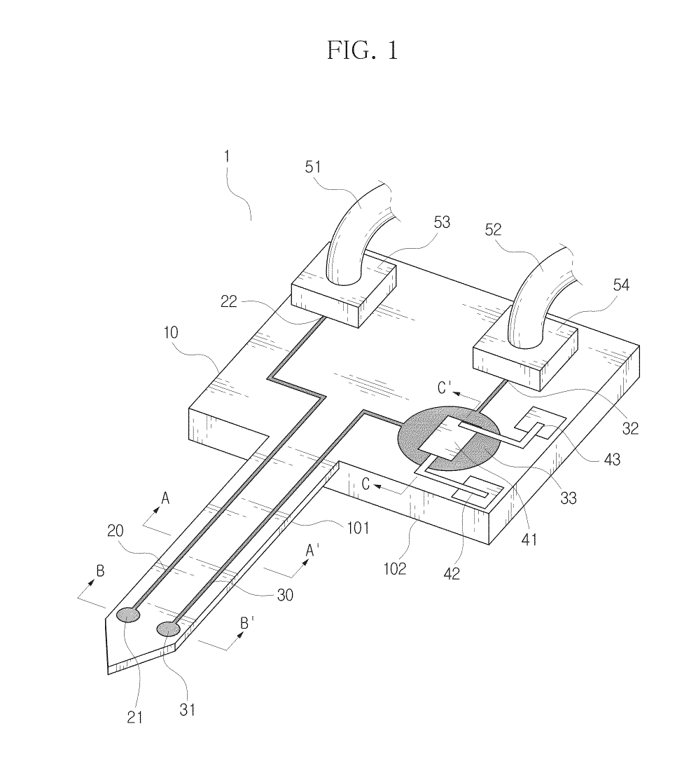

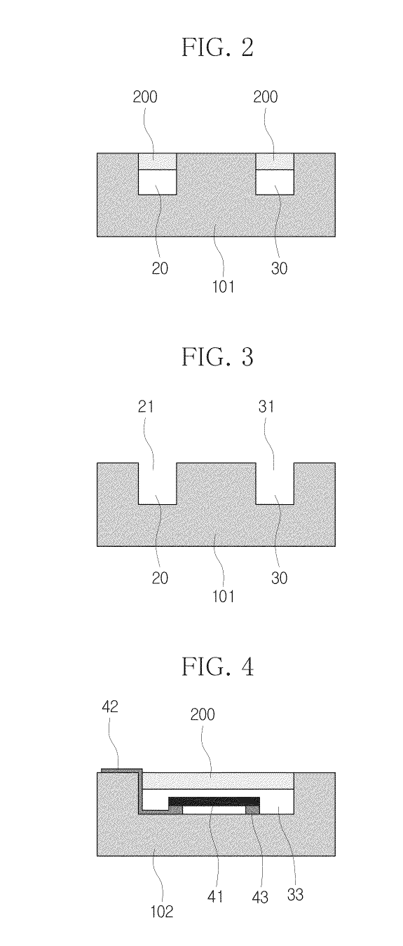

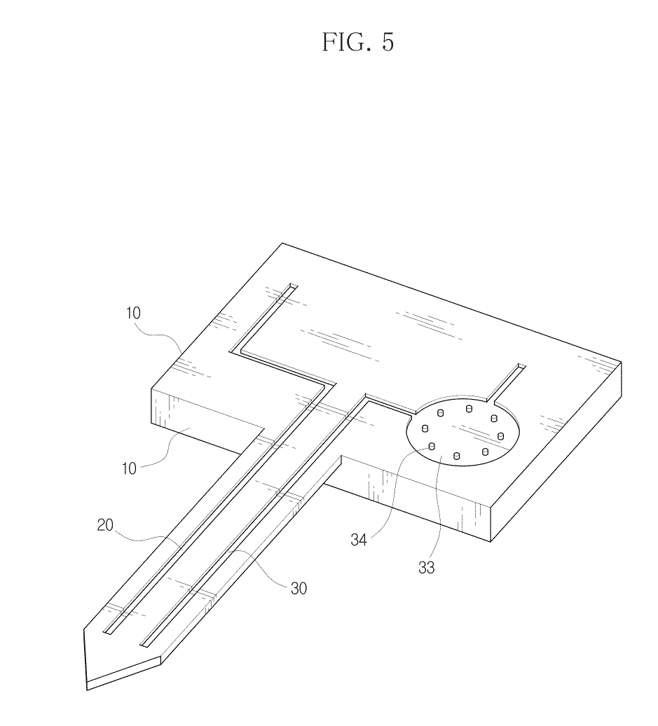

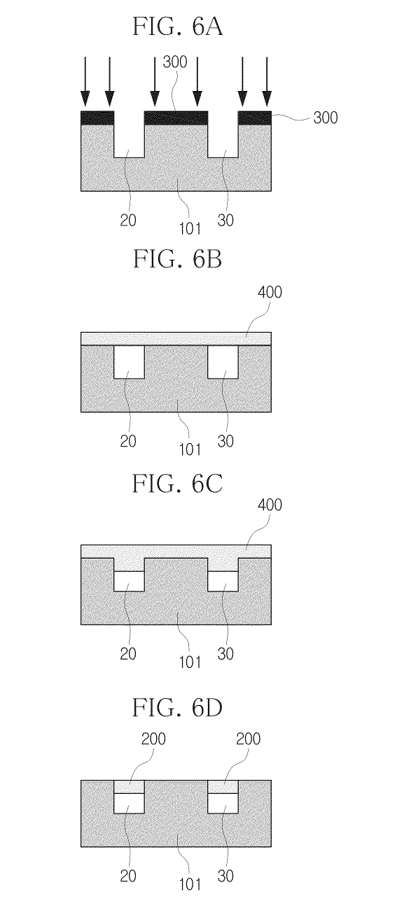

FIG. 1 is a perspective view showing a micro sensing system 1 according to an embodiment of the present disclosure, FIG. 2 is a cross-sectional view, taken along the line A-A′ of FIG. 1, FIG. 3 is a cross-sectional view, taken along the line B-B′ of FIG. 1, and FIG. 4 is a cross-sectional view, taken along the line C-C′ of FIG. 1.

As shown in FIG. 1, the micro sensing system 1 includes a body 10, a neurotrophic factor channel 30 and a buffer solution channel 20 formed in the body 10 to allow a fluid flow therein, a storage unit 33 formed on a path of the neurotrophic factor channel 30 in the body 10, and a biosensor 41 integrated in the storage unit 33 to sense a neurotrophic factor.

The body 10 includes a probe body 101 having a sharp tip and extending thin and long so as to be inserted into a living body having a body fluid containing neurotrophic factors, and a main body 102 formed at the rear of the probe body 101.

The buffer solution channel 20 extends from a portion near the front end of the probe body 101, extends along a length direction of the probe body 101, is bent once at the main body 102, and extends to a portion near the rear end of the main body 102. A buffer solution outlet 21 is formed at the front end of the buffer solution channel 20 to open toward the top of the probe body 101, and though not shown in detail, the buffer solution inlet 22 is formed at the rear end to open toward the top of the main body 102.

At the rear end of the buffer solution channel 20 having the buffer solution inlet 22, the fluid inlet tube 51 is coupled to the top of the main body 102 through the channel connector 53. The buffer solution inlet 22 and the fluid inlet tube 51 are communicated with each other to allow a fluid to flow.

The neurotrophic factor channel 30 extends from a portion near the front end of the probe body 101, extends along a length direction of the probe body 101, is bent once at the main body 102 in a direction opposite to the buffer solution channel 20, and extends to a portion near the rear end of the main body 102. A neurotrophic factor inlet 31 is formed at the front end of the neurotrophic factor channel 30 to open toward the top of the probe body 101, and though not shown in detail, the neurotrophic factor outlet 32 is formed at the rear end to open toward the top of the main body 102.

At the rear end of the neurotrophic factor channel 30 having the neurotrophic factor outlet 32, the fluid outlet tube 52 is coupled to the top of the main body 102 through the channel connector 54. The neurotrophic factor outlet 32 and the fluid outlet tube 52 are communicated with each other to allow a fluid to flow.

As shown in FIG. 2, the buffer solution channel 20 and the neurotrophic factor channel 30 are formed along the inside of the body 10. As described later, the buffer solution channel 20 and the neurotrophic factor channel 30 are prepared by forming a groove concavely etched in the top surface of the body 10 by means of deep reactive ion etching (DRIE) and coupling a cover 200 to a top opening of the etched groove.

However, a micro fluid channel may also be formed in the body 10 by means of physical or chemical drilling, without being limited to the above method.

Meanwhile, when the cover 200 is removed at a portion of the buffer solution channel 20 and the neurotrophic factor channel 30, the buffer solution outlet 21 and the neurotrophic factor inlet 31 may be formed as shown in FIG. 3.

Even though FIG. 1 shows that the buffer solution outlet 21 and the neurotrophic factor inlet 31 have a circular shape with a greater width than the buffer solution channel 20 and the neurotrophic factor channel 30, the present disclosure is not limited thereto, and the buffer solution outlet 21 and the neurotrophic factor inlet 31 may also be formed to have the same width as the buffer solution channel 20 and the neurotrophic factor channel 30 as shown in FIG. 3.

Referring to FIGS. 1 and 4, the storage unit 33 having a diameter relatively greater than the width of the neurotrophic factor channel 30 is formed on a path of the neurotrophic factor channel 30 in the main body 10.

As well shown in FIG. 4, the storage unit 33 is formed by a groove concave in the top of the body 10.

Electrodes 42, 43 exposed toward the top surface of the main body 102 are fixed in the storage unit 33, and a biosensor 41 is coupled to the electrodes 42, 43. The cover 200 is coupled to the opened top of the storage unit 33 to close the storage unit 33.

The biosensor 41 of this embodiment is a subminiature sensor and may be selected from a chemical sensor, an electric sensor and a resonance sensor, which may sense a neurotrophic factor in a solution.

The signal detected by the biosensor 41 may be transferred to the outside through the electrodes 42, 43 and transmitted to various analysis devices.

Various kinds of chemical sensors, electric sensors and resonance sensors are already known in the art, and the principle of sensing a neurotrophic factor by the biosensor is beyond the technical spirit of the present disclosure and is not described in detail here.

Hereinafter, operations of the micro sensing system 1 of this embodiment will be described with reference to FIG. 1.

The micro sensing system 1 of this embodiment is inserted into the brain to observe a change of concentration of brain neurotrophic factors in the brain fluid and thus reveals brain neurotrophic factors in relation to behaviors and diseases of a specific animal.

First, the probe body 101 is inserted into a desired position of the brain. The insertion position may be a brain part which has been proved as causing a neurotrophic factor in relation to a specific behavior or disease.

The probe body 101 is at least partially inserted into the brain, and at least the buffer solution outlet 21 and the neurotrophic factor inlet 31 are inserted into the brain.

Next, a buffer solution is forced to flow in through the fluid inlet tube 51 from the outside by a strong pressure. In this embodiment, the buffer solution employs a saline solution.

The buffer solution is introduced into the brain by flowing into the buffer solution channel 20 through the buffer solution inlet 22, flowing along the channel, and outputting through the buffer solution outlet 21.

By injecting the buffer solution into the brain by a strong pressure, the brain pressure is locally increased, and the brain fluid in the brain flows into the neurotrophic factor channel 30 having a relatively low pressure through the neurotrophic factor inlet 31.

As the buffer solution is continuously injected, the brain fluid introduced into the neurotrophic factor channel 30 flows along the neurotrophic factor channel 30.

The brain fluid flowing along the neurotrophic factor channel 30 fills the storage unit 33 having a relatively great volume, is stored in the storage unit 33, discharges from the storage unit 33, and discharges to the fluid outlet tube 52 through the neurotrophic factor outlet 32.

The brain fluid flowing out from the fluid outlet tube 52 is collected in an external storage unit (not shown).

Meanwhile, the brain fluid filling the storage unit 33 directly contacts the biosensor 41, and the biosensor 41 senses a change of concentration of a neurotrophic factor included in the brain fluid.

The sensing information of the biosensor 41 is transferred to an external analysis device through the electrodes 42, 43.

In the micro sensing system 1 of this embodiment, the biosensor 41 is installed in the storage unit 33 formed on a path of the neurotrophic factor channel 30. Therefore, it is possible to collect a sufficient amount of neurotrophic factor and sense a change of concentration of the neurotrophic factor within a short time in comparison to an existing technique in which the brain fluid should be extracted to the external storage unit and a sufficient amount of brain fluid is collected for a long time.

In addition, since the biosensor 41 is directly attached to the body, the brain from which the brain fluid is extracted is very close to the sensor, and since the biosensor 41 directly contacts the brain fluid stored in the storage unit 33, the biosensor 41 may be continuously exposed to the flowing brain fluid. Therefore, a change amount of the neurotrophic factor in the brain fluid may be measured in real time.

Meanwhile, since the probe body is inserted into the brain to extract the brain fluid, the sensor system should have a small size. In this embodiment, a subminiature sensor system having a very small size may be formed using a micromachining technique and a reflow process.

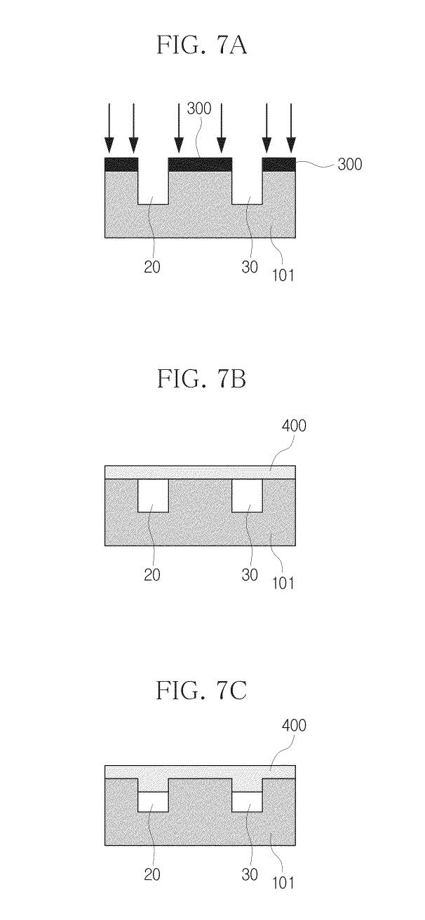

FIGS. 5 to 8 are diagrams for illustrating a method for forming the micro sensing system 1.

As shown in FIG. 5, first, a silicon wafer is provided as the body 10, and the channels 20, 30 and the storage unit 33 are formed simultaneously on the body 10.

Even though FIG. 5 shows that the body 10 has a shape to form the probe body and the main body, this is just for convenience, and it should be understood that the channels 20, 30 and the storage unit 33 are formed on a bulk wafer, a single cover 400 having a plate shape is adhered to the entire top surface of the wafer, and then the bulk wafer is etched to finally form the shape of the body 10.

As shown in FIG. 5, grooves for forming the micro fluid channels 20, 30 and the storage unit 33 are concavely etched at the top surface of the body 10. The grooves for forming the micro fluid channels 20, 30 and the storage unit 33 are prepared by means of the DRIE process.

As described later, the cover 200 is formed by melting a cover made of glass material and partially depressing the glass at the top surface of the etched groove by means of a reflow process. Therefore, in the storage unit 33 having a relatively great area, a plurality of protrusions 34 serving as pillars to prevent the cover 200 from collapsing is formed.

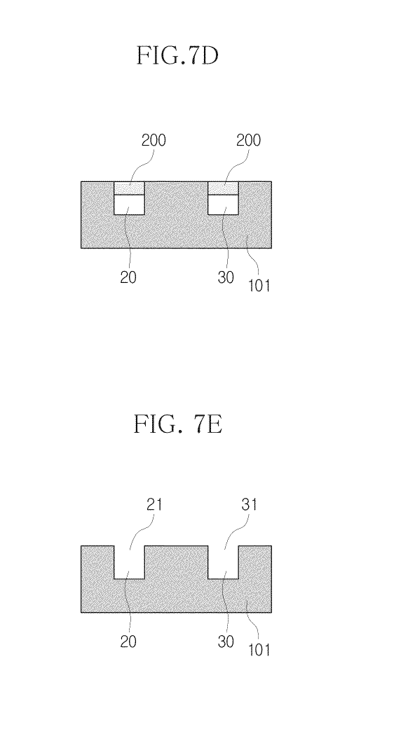

First, referring to FIGS. 6A to 6D, the method for forming the structure depicted in FIG. 2 will be described.

As shown in FIG. 6A, a first mask 300 is applied other than regions where the buffer solution channel 20 and the neurotrophic factor channel 30 are to be formed.

In a state where the first mask 300 is applied, the DRIE process is performed to form grooves of a predetermined depth, and then the first mask 300 is removed.

Next, as shown in FIG. 6B, a flat substrate 400 made of a glass material is coupled to the top surface of the body 10 having the grooves in a vacuum state. In this embodiment, the body 10 and the substrate 400 are strongly adhered to each other by means of anodic bonding which is a coupling method using a voltage. The substrate 400 made of a glass material has a lower softening point in comparison to the body 10 made of a silicon material.

As the body 10 and the substrate 400 are adhered, the grooves formed in the top of the body 10 are closed and sealed in a vacuum state by means of the substrate 400.

After that, as shown in FIG. 6C, the body 10 and the substrate 400 adhered to each other are put into a furnace (not shown) in a non-vacuum state and is heated at a temperature higher than a melting point of glass and lower than a melting point of silicon.

Accordingly, the substrate 400 melted softly flows down into the grooves. At this time, there is a predetermined difference in pressure between the grooves in a vacuum state and the outer regions in a non-vacuum state, and the melted substrate 400 is sucked into the grooves due to this difference in pressure to fill the grooves more rapidly and effectively.

If the substrate 400 flows into the grooves to a predetermined depth, the heat is intercepted to harden the substrate 400 again.

After that, as shown in FIG. 6D, if the substrate 400 is sufficiently hardened, the top portion of the substrate 400 located on the top surface of the body 10 is removed to form the cover 200 which is flat with the top surface of the body 10 and closes the channels 20, 30.

Since the cover 200 depressed toward the inside of the body 10 may be formed through the reflow process in which glass is melted, the thickness of the probe body 101 having the channels 20, 30 may be greatly reduced, which may decrease a damage of the brain into which the probe body 101 is inserted.

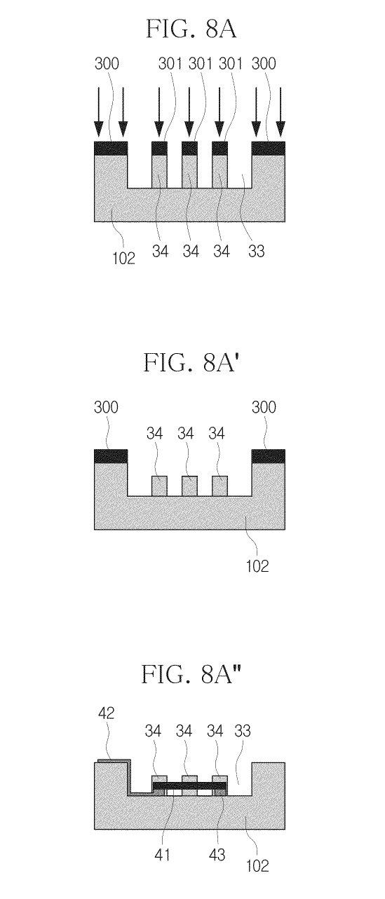

FIG. 7A to 7E are diagrams for illustrating a method for forming the structure of FIG. 3.

Even though FIGS. 7A to 7E are provided separate from FIGS. 6A to 6D for convenience, the processes of FIGS. 7A to 7D are substantially identical to the processes of FIGS. 6A to 6D.

However, as shown in FIG. 7E, a process of drilling the top portion of the formed cover 200 to form the buffer solution outlet 21 and the neurotrophic factor inlet 31 is additionally performed.

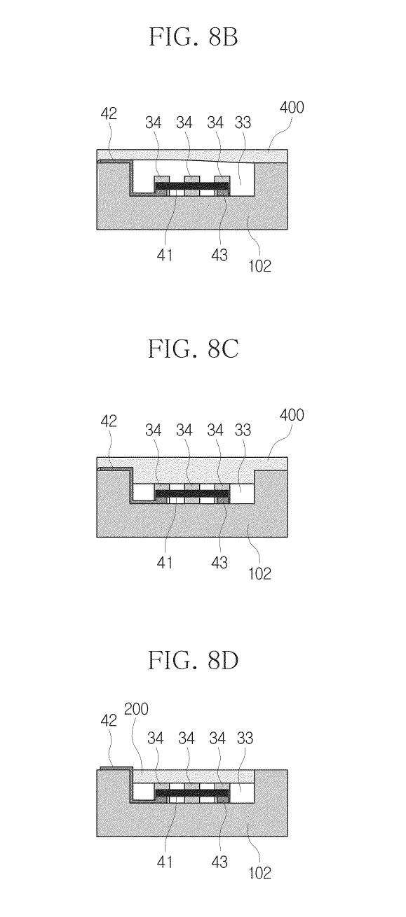

FIGS. 8A, 8A′, 8A″, 8B to 8D are diagrams for illustrating a method for forming the structure of FIG. 4. In FIG. 8, a protrusion 34 observed at the rear of the biosensor 41 is depicted together for convenience.

As shown in FIG. 8A, a second mask 301 is applied to a portion where the protrusion 34 is to be formed, in addition to the first mask 300.

In a state where both the first mask and the second mask are applied, the DRIE process is performed to form a groove in which the protrusion 34 is formed.

If a groove of a predetermined depth is formed, as shown in FIG. 8A′, the second mask 301 is removed, and a secondary DRIE process is performed.

Through the secondary DRIE process, the entire groove having the protrusion 34 is further etched in the depth direction. If the secondary DRIE process is performed, the protrusion 34 has a height lower than the entire depth of the groove.

Next, as shown in FIG. 8A″, the electrodes 42, 43 are attached in the groove to avoid the protrusion 34, and the biosensor 41 is attached onto the electrodes 42, 43. The height where the biosensor 41 is disposed is lower than the height of the protrusion 34.

Next, as shown in FIG. 8B, the flat substrate 400 made of a glass material is coupled to the top surface of the body 10 having the groove in a vacuum state.

At this time, since the electrode 41 is attached to the top surface of the body 10, the substrate 400 may not be adhered to the body 10 at a portion near the electrode 41. However, since a non-adhered portion has an area very smaller than the area of the body 10 and the substrate 400, the groove may be made vacuous not difficultly.

After that, as shown in FIG. 8C, the body 10 and the substrate 400 adhered to each other are put into a furnace (not shown) in a non-vacuum state and are heated at a temperature higher than a melting point of glass and lower than a melting point of silicon.

Accordingly, the substrate 400 melted softly flows down into the grooves. At this time, there is a predetermined difference in pressure between the grooves in a vacuum state and the outer regions in a non-vacuum state, and the melted substrate 400 is sucked into the grooves due to this difference in pressure to fill the grooves more rapidly and effectively.

Since the groove forming the storage unit 33 has a relatively great area, if the melting process is delayed, the substrate 400 may be unexpectedly melted and close the groove.

In this embodiment, since the plurality of protrusions 34 is formed at various positions of the groove, the protrusion 34 may prevent the substrate 400 from collapsing into the entire groove by blocking the substrate 400 rapidly collapsing to the lower portion of the groove.

After that, as shown in FIG. 8D, the top portion of the substrate 400 is polished so that the electrodes 41, 43 are exposed to the top surface of the body 10.

In this embodiment, since the channels and the storage unit are formed simultaneously by means of a deep reactive etching process and the cover is formed by means of a reflow process, it is possible to form a precise sensor system with a very small size.

What is claimed is:

1. A micro sensing system for sensing a neurotrophic factor included in a body fluid, the micro sensing system comprising:
a body;
a neurotrophic factor channel formed in the body to allow a fluid flow therein; and
a biosensor formed at the body to sense the neurotrophic factor,
wherein the body fluid is extracted in a living body through the neurotrophic factor channel, and
wherein the biosensor is disposed on a path of the neurotrophic factor channel to directly contact a body fluid flowing through the neurotrophic factor channel and senses a concentration of a neurotrophic factor in the body fluid.

2. The micro sensing system according to claim 1,
wherein the body includes a storage unit formed on a path of the neurotrophic factor channel to store the body fluid flowing along the neurotrophic factor channel, and
wherein the biosensor is integrated in the storage unit.

3. The micro sensing system according to claim 2,
wherein the neurotrophic factor channel and the storage unit are formed simultaneously by means of deep etching.

4. The micro sensing system according to claim 2,
wherein a cover is provided to close upper portions of the neurotrophic factor channel and the storage unit, and
wherein a plurality of protrusions for preventing the cover from collapsing is formed at the storage unit.

5. The micro sensing system according to claim 1, further comprising a buffer solution channel formed in the body to allow a buffer solution to flow into the living body,
wherein a pressure in the living body increases by the buffer solution flowing into the living body, and thus the body fluid flows into the neurotrophic factor channel.

6. The micro sensing system according to claim 1, wherein the body includes:
a probe body extending to be inserted into the living body; and
a main body formed at a rear end of the probe body to be located out of the living body,
wherein the neurotrophic factor channel extends from the probe body to the main body, and
wherein the biosensor is disposed at the main body.

7. The micro sensing system according to claim 1,
wherein the biosensor senses in real time a concentration of the neurotrophic factor in the body fluid which continuously flows in the neurotrophic factor channel.

8. The micro sensing system according to claim 1,
wherein the biosensor is a chemical sensor, an electric sensor or a resonance sensor.
